(19) United States
(12) Patent Application Publication
(10) Pub. No.: US 2026/0324982 A1
(43) Pub. Date: Oct. 22, 2026

(54) SYSTEMS AND METHODS FOR OPTICAL AND ACOUSTIC COUPLING MATERIALS, SYSTEMS, AND METHODS OF USE

(71) Applicant: Arizona Board of Regents on Behalf of the University of Arizona, Tucson, AZ (US)

(72) Inventors: Russell Witte, Tucson, AZ (US); Christopher Salinas, Tucson, AZ (US); Eric Reichel, Tucson, AZ (US)

(21) Appl. No.: 19/119,513

(22) PCT Filed: Oct. 10, 2023
(86) PCT No.: PCT/US23/76492

§ 371 Date: Apr. 9, 2025

Related U.S. Application Data
(60) Provisional application No. 63/378,976, filed on Oct. 10, 2022.

(51) Int. Cl. A61B 5/00

(52) U.S. Cl. CPC A61B 5/0097; A61B 5/4064; A61B 2562/146

(57) ABSTRACT

A system includes an adapter for a photoacoustic imaging system that includes a coupling media made from gellan gum, which exhibits improved acoustic and optical properties for facilitation of photoacoustic (PA) and pulse-echo (PE) imaging. The system is particularly aimed at improving imaging within a short-wave infrared (SWIR) frequency band. The coupling media can include deuterium oxide mixed with gellan gum for further improvement of PA and PE imaging. Validation studies are presented herein for comparison of gellan gum-based coupling media with currently-available coupling media.

### CROSS-REFERENCE TO RELATED APPLICATIONS

This is a PCT Patent Application that claims benefit to U.S. Provisional Patent Application Ser. No. 63/378,976 filed Oct. 10, 2022, which is herein incorporated by reference in its entirety.

### GOVERNMENT SUPPORT

This invention was made with government support under AR074627 awarded by National Institutes of Health. The government has certain rights in the invention.

### FIELD

The present disclosure generally relates to materials, compositions, and devices for use with systems and methods of photoacoustic imaging as well as monitoring therapy.

### BACKGROUND

Biomedical photoacoustic imaging (PAI) is a high-resolution imaging modality that uses the optical properties of a material to gain image contrast. Exposure of tissue to short-pulsed light generates ultrasound (US) through optical absorption, where these acoustic waves can be imaged by traditional US means. However, there remain fundamental challenges with efficiently and optimally delivering light and sound through coupling materials that are placed between a device that emits and/or detects light and/or sound.

It is with these observations in mind, among others, that various aspects of the present disclosure were conceived and developed.

### SUMMARY

According to various aspects, the present disclosure relates to a coupling device for imaging configured to efficiently propagate light and sound. In particular, the coupling device is configured for optical imaging with ultrasound modulation, such as wherein the optical imaging is laser speckle doppler imaging combined with ultrasound neuromodulation.

In another aspect, the present disclosure relates to photoacoustic adapter for use with an ultrasonic probe. The adapter can include a proximal portion configured to engage the ultrasonic probe, a distal portion configured to contact a sample surface, and a coupling medium chamber disposed between the proximal portion and the distal portion. The coupling medium chamber includes a coupling medium.

According to one aspect, the adapter is an inline adapter configured to co-align optical transmissions from the ultrasonic probe and acoustic emissions received at the ultrasonic probe. In another aspect, the coupling medium includes a water-based gelling agent. The water-based gelling agent can include low-acyl Gellan gum.

In yet another aspect, the coupling medium includes deuterium oxide (“heavy water”) and a gelling agent. The gelling agent can include low-acyl Gellan gum.

The present disclosure also relates to a photoacoustic reflection apparatus for real-time optical monitoring using ultrasound. In one aspect, the apparatus includes a housing that defines a photoacoustic reflection chamber. The housing can also include a first optical aperture defined in a first surface, a second optical aperture defined in a second surface, where the second surface is disposed opposite the first surface, and the first and second optical apertures are coaligned along a first axis. The housing also defines an ultrasound transducer port in a third surface that is substantially parallel to the first axis such that acoustic emissions from an ultrasound transducer are perpendicular to the first axis. The apparatus also includes a coupling medium disposed within the housing and a prism reflector affixed within the housing, where the acoustic emissions contact the prism reflector and are reflected along the first axis towards a sample aligned with the second optical aperture. A response of the sample is observable by an optical sensor aligned with the first optical aperture.

In one aspect, the coupling medium includes water. In another aspect, the coupling medium includes deuterium oxide. In yet another aspect, the coupling medium includes a mixture of a transparent gelling agent and water or a mixture of Gellan gum and deuterium oxide.

The present disclosure also relates to an ultrasonic coupling medium for use with an ultrasonic probe. The coupling medium includes a mixture of Gellan gum and deuterium oxide. In one aspect, the mixture contains Gellan gum in a range between 1.5% w/w and 3% w/w.

Further, the present disclosure relates to a method of fabricating a coupling medium. The method includes steps such as pre-heating deuterium oxide, porting gellan gum into the deuterium oxide, stirring the resultant mixture until the gellan gum dissolves into the deuterium oxide, degassing the mixture to remove bubble artifacts, pouring the mixture into a molding apparatus, and chilling the mixture until the mixture forms a gel in the shape of the molding apparatus.

### BRIEF DESCRIPTION OF THE DRAWINGS

FIGS. 1A and 1B are a pair of laser speckle images of a brain “before” and “after” before and after TBI that shows a significant reduction in blood flow post-TBI;

FIG. 2A is an illustration showing a system for real-time photoacoustic imaging that includes a photoacoustic adapter for use with an ultrasonic probe and an optical sensor;

FIG. 2B is an illustration showing a system for real-time photoacoustic imaging that includes an “inline” photoacoustic adapter for use with an ultrasonic probe;

FIG. 2C is an image showing one embodiment of the photoacoustic adapter of FIG. 2B;

FIGS. 3A-3C are a series of images taken by an optical sensor (e.g., the optical sensor of FIG. 2A) during therapeutic ultrasound treatment;

FIGS. 4A-4D are a series of graphical representations showing blood perfusion of an object of interest during therapeutic ultrasound treatment;

FIG. 5 is a graphical representation showing absorption coefficient for water and lipids across different wavelengths of light;

FIGS. 6A-6C is an illustration of various experimental setups used to determine the speed of sound and acoustic attenuation, according to various embodiments;

FIG. 7 is an illustration of an expected baseline photoacoustic signal measured with an acoustic microscope and an acoustic microscope setup testing the optical clarity of each coupling material, according to various embodiments;

FIG. 7A shows an acoustic microscope setup for testing optical clarity of each coupling material;

FIG. 7B shows an expected baseline photoacoustic signal measured with an acoustic microscope;

FIG. 8 is a graphical representation showing acoustic attenuation for various coupling materials including gellan gum;

FIGS. 9A-9D are a series of photoacoustic images of an 800 μm Gaussian spot through each material;

FIG. 9E is a graphical representation showing a “cross-sectional slice” of the relative photoacoustic amplitudes for baseline, gellan gum, Humimic, and agarose, corresponding with FIGS. 9A-9D;

FIGS. 10A and 10B are a pair of illustrations respectively showing transmission-mode and reflection-mode setups for quantification of optical loss and acoustic propagation of gellan gum as a coupling media;

FIGS. 11A and 11B is a graphical representation showing transmission-mode data (absorption plots) for quantification of optical loss for both heavy water gellan gum and distilled water gellan gum;

FIG. 12 is a graphical representation showing axial spatial resolution for pulse-echo images obtained from a sample with fine graphite particles using humimic, heavy water gellan gum and distilled water gellan gum as coupling media;

FIG. 13 is a sequence of photoacoustic images at various wavelengths for both heavy water gellan gum and distilled water gellan gum;

FIG. 14 is a graphical representation showing photoacoustic surface spectra for samples imaged using heavy water gellan gum and distilled water gellan gum, demonstrating signals above noise for heavy water gellan gum across the full range, including distinct peaks of lipid/water;

FIGS. 15A-15C display a PA imaging cross-section of a bovine tissue sample along with spectral data captured using heavy water gellan gum as coupling media; and

FIG. 16 shows a method for fabrication of a coupling medium including heavy water and gellan gum.

Corresponding reference characters indicate corresponding elements among the view of the drawings. The headings used in the figures do not limit the scope of the claims.

### DETAILED DESCRIPTION

The present disclosure relates to materials, compositions, and devices with novel/optimal optical and acoustic properties that enable or improve systems and methods of coupling of light and sound for biomedical and defense applications related to imaging (e.g., photoacoustic) and monitoring therapy (e.g., image-guided ultrasound neuromodulation). To achieve this goal, it is important to understand and solve different challenges described herein.

### 1. Overview and Motivations

Photoacoustic imaging (PAI) is a powerful screening tool for cancer detection; capable of mapping vasculature, measuring blood oxygenation, and providing material specificity. For clinical applications such as in vivo skin imaging, PAI laser systems must be highly efficient in light delivery. Several types of media have been proposed for coupling light and sound from a laser to a tissue interface. One approach combines an ultrasound (US) probe with an inline adapter using a photoacoustic prism/reflector filled with water, agarose gel or optically transparent rubber for coupling. However, adapter efficiencies are hindered by suboptimal material properties (optical/acoustic), all which degrade image quality (e.g., artifacts, lower signal-to-noise ratios, etc.).

Biomedical PAI is a high-resolution imaging modality that uses the optical properties of a material to gain image contrast. Exposure of tissue to short-pulsed light generates US through optical absorption, where these acoustic waves can be imaged by traditional US means. By taking advantage of endogenous or exogenous contrast agents, PAI can provide tissue specificity, making it an excellent candidate for non-invasive cancer screening. PAI however does not come without its challenges, as the dependance upon optical and acoustic tissue properties can require complex system designs. For clinical applications such as in vivo skin imaging, it is common to combine light and sound with an inline reflector, which coaxially aligns the optical and acoustic paths within a single coupling media. In this approach, the coupling media plays a crucial role as it must maximize light transmission to tissue while minimizing ultrasound attenuation. Furthermore, in systems such as photoacoustic microscopes, incident light must remain unaberrated, requiring minimal optical scattering. Finally, an ideal coupling media must have similar mechanical properties (speed of sound, acoustic impedance, etc.) to tissue to prevent reflection losses at interfaces.

Common Photoacoustic (PA) coupling media such as water, oil-based rubbers, or agarose gel (AG) partially meet these requirements, however each lack in their own regard. Water has high optical and acoustic clarity but is prone to leakage and can introduce artifacts due to bubbles in the liquid. Oil-based rubbers can maintain their structure and possess a long shelf life. However, these materials tend to be more acoustically attenuating, particularly towards high frequency ultrasound. Lastly, AG couples well to tissue with minimal acoustic attenuation but increase overall optical scattering. The present disclosure relates to the use low acyl gellan gum (GG) for PA coupling as it is both optically and acoustically clear and demonstrated high durability as a coupling media.

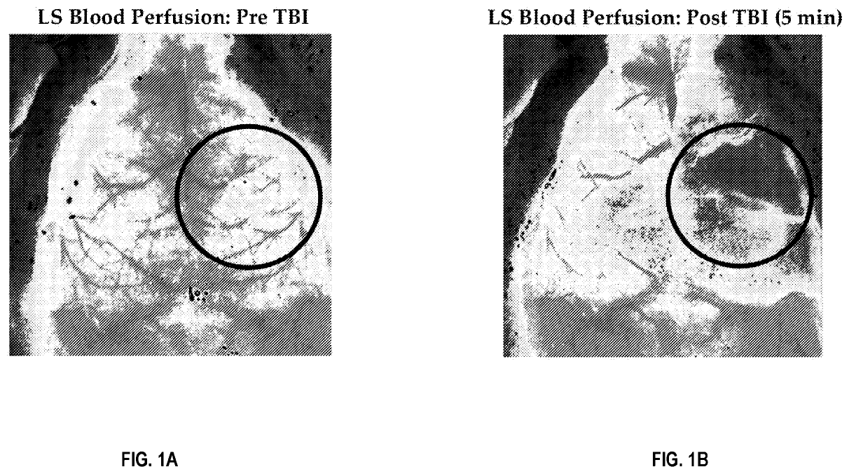

As such, the systems outlined herein, including a photoacoustic reflection apparatus and coupling media, enable real-time monitoring of organ function during therapeutic ultrasound. An embodiment of the system is presented herein in the context of therapeutic transcranial ultrasound (e.g., low-intensity pulsed ultrasound (LIPUS)), which has been shown to decrease recovery time from traumatic brain injury (TBI). It is hypothesized that transcranial LIPUS improves functional prognosis after TBI, increases blood perfusion, reduces edema, and is neuroprotective. However, neuroprotective mechanisms of transcranial ultrasound are not well-understood. Factors of interest during therapeutic ultrasound include hemodynamics, neuronal activity, oxygen saturation, and temperature. For example, FIG. 1A shows laser speckle images of a brain before and after TBI that shows a significant reduction in blood flow in the circled region of FIG. 1B. When used along with therapeutic ultrasound treatment, imaging systems can look at snapshots before and after treatment as in FIGS. 1A and 1B but are considerably limited in providing real-time monitoring of hemodynamics and other factors.

### 2. Photoacoustic Adapter

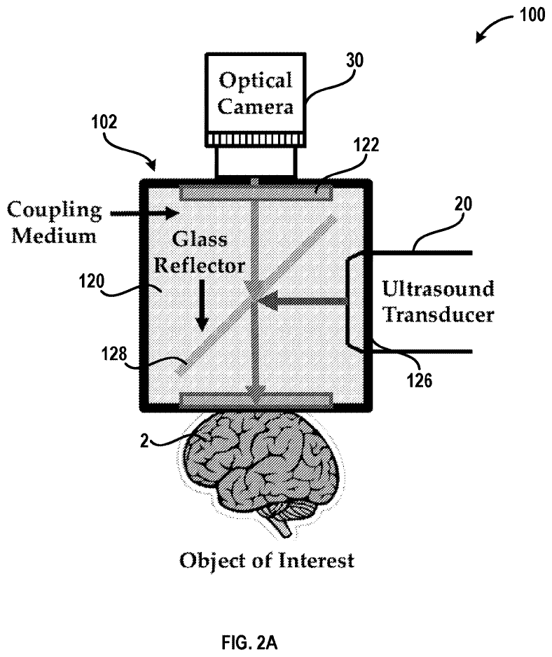

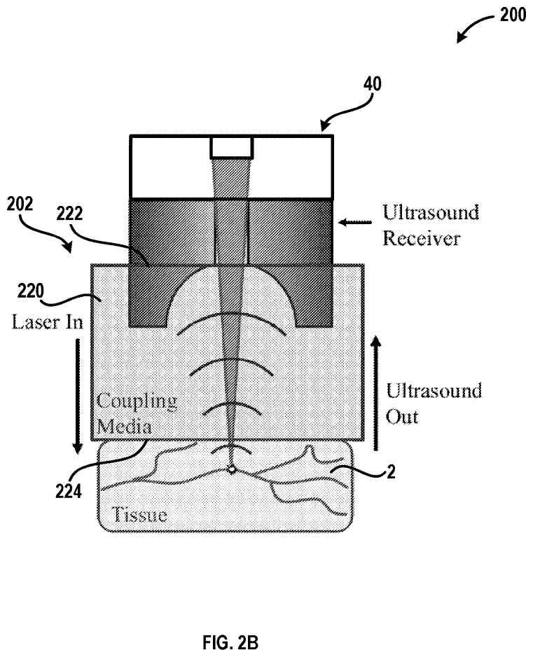

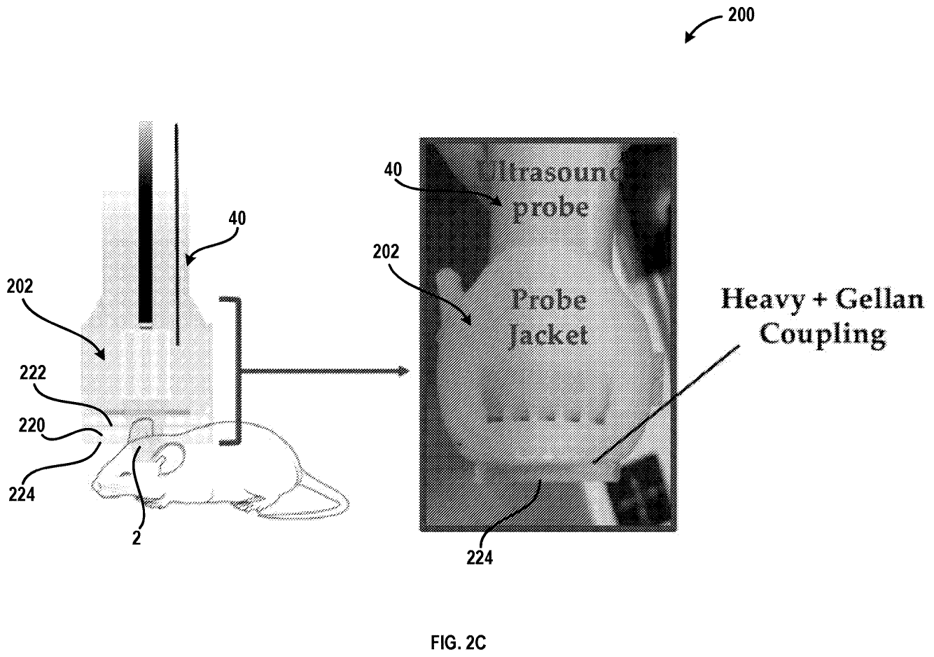

FIG. 2A is an illustration of one embodiment of a photoacoustic adapter defining an acoustic reflection window that enables monitoring of the real-time physical response to application of ultrasound. The apparatus may be configured for use with existing ultrasound transducers (e.g., ultrasound probe 20) and optical sensors or cameras (e.g., imaging device 30) to provide real-time observation and monitoring therapy among others uses.

FIG. 2A shows a first system 100 for real-time photoacoustic imaging that includes a photoacoustic adapter 102 for use with an ultrasonic probe 20 (or “ultrasound transducer”) and an optical sensor 30. The photoacoustic adapter 102 includes a coupling medium chamber 120 having a coupling medium that engages the ultrasonic probe 20 and contacts a surface of a sample.

The coupling medium chamber 120 can include a first optical window 122 and a second optical window 124 defined opposite from the first optical window 122. The first optical window 122 can be associated with the optical sensor 30 and the second optical window 124 can contact an object of interest 2 (e.g., a sample such as in-vivo tissue). The coupling medium chamber 120 can include an ultrasound transducer port 126 that engages the ultrasound transducer 20. The first optical window 122 and the second optical window 124 can be coaligned along a first axis and the ultrasound transducer port 126 can be aligned along a second axis that is perpendicular to the first axis as shown. The coupling medium chamber 120 can include a prism element 128 positioned therein that reflects acoustic emissions from the ultrasound transducer 20 towards the object of interest 2, e.g., altering a path of the acoustic emissions to align with the first axis. The optical sensor 30 can observe a response of the object of interest 2 resultant of the acoustic emissions from the ultrasound transducer 20.

FIG. 2B shows an illustration of a second system 200 including a photoacoustic adapter 202 for use with an ultrasonic probe 40. Where the example of FIG. 2B is applied in the context of photoacoustic imaging, the ultrasonic probe 40 can deliver optical transmissions (e.g., a laser) to the object of interest 2 and can observe acoustic emissions from the object of interest 2 resultant of the light from the ultrasonic probe 40. The photoacoustic adapter 202 can be thus configured to co-align optical transmissions from the ultrasonic probe 40 and acoustic emissions received from the object of interest 2 at the ultrasonic probe 40. Alternatively, the ultrasonic probe 40 can be a standard ultrasound probe that applies acoustic emissions to the object of interest 2 and receives acoustic emissions from the object of interest 2 in response.

The photoacoustic adapter 202 can likewise include a coupling medium chamber 220 filled with a coupling medium (e.g., coupling media with gellan gum as outlined in section 3 or section 4 herein) and defining a proximal portion 222 and a distal portion 224 opposite from the proximal portion 222. The proximal portion 222 can be associated with the photoacoustic probe 40 and the distal portion 224 can contact the object of interest 2 (e.g., a sample such as in-vivo tissue). Light from the ultrasonic probe 40 enters the coupling medium chamber 220 through the proximal portion 222, propagates through the coupling medium, and exits the coupling medium chamber 220 through the distal portion 224. Acoustic emissions from the object of interest 2 enter the distal portion 224, propagate through the coupling medium, and exit the proximal portion 222 for receipt at the ultrasonic probe 40.

FIG. 2C shows a version of the photoacoustic adapter 202 for reflection-mode SWIR, embodied as a “probe jacket” that covers a distal portion of the ultrasonic probe 40 and positions the coupling media at the distal portion of the ultrasonic probe 40. As shown, the distal portion 224 of the coupling medium chamber 220 contacts the object of interest 2.

The coupling medium disposed within the coupling medium chamber 120 (FIG. 2A) or 220 (FIG. 2B) can include a gelling agent, which can include gellan gum. The coupling medium can further include hydrogen oxide (H₂O, or “water”) or can alternatively include deuterium oxide (D₂O, or “heavy water” which is an isotope of water). A first example implementation validated in section 3 herein includes a coupling medium having gellan gum and deionized hydrogen oxide, and is compared with other coupling media including hydrogen oxide (without gellan gum), agarose and humimic medical gelatin. A second example implementation validated in section 4 herein includes a coupling medium having gellan gum and deuterium oxide, and is compared with other coupling media including gellan gum with hydrogen oxide as in the first example implementation, pure deuterium oxide, pure hydrogen oxide, and humimic medical gelatin.

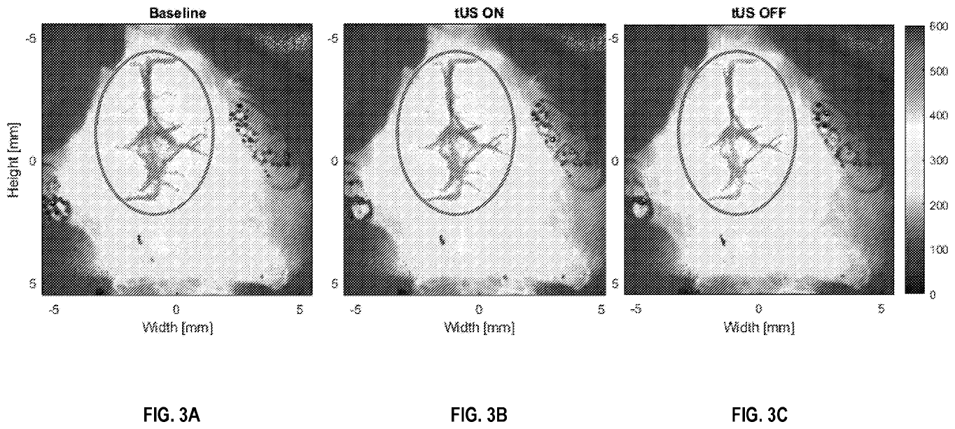

FIGS. 3A-3C show a sequence of images taken by an optical sensor such as the optical sensor 20 of FIG. 2A during therapeutic ultrasound treatment (as opposed to “before” and “after” as discussed above). FIG. 3A shows a “baseline” image for Pre-US intervention (averaged frames before application of therapeutic ultrasound), FIG. 3B shows an image with averaged frames taken during application of therapeutic ultrasound, and FIG. 3B shows an image with averaged frames taken after application of therapeutic ultrasound.

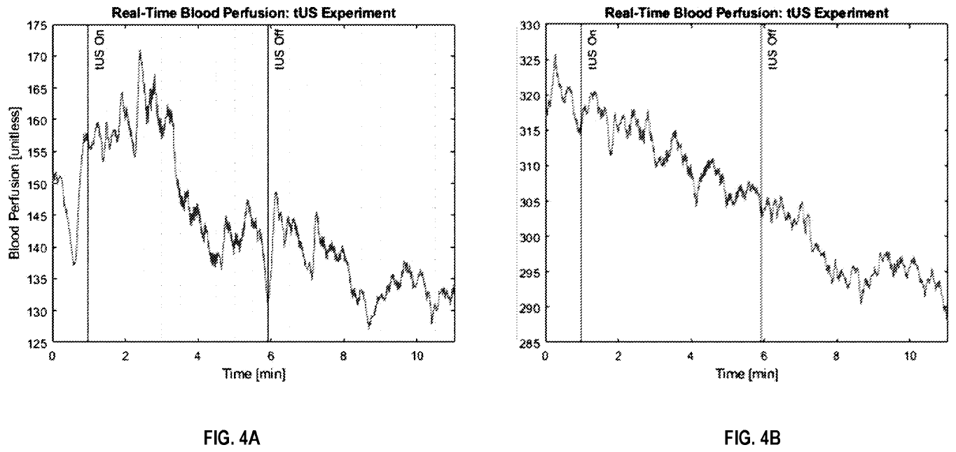

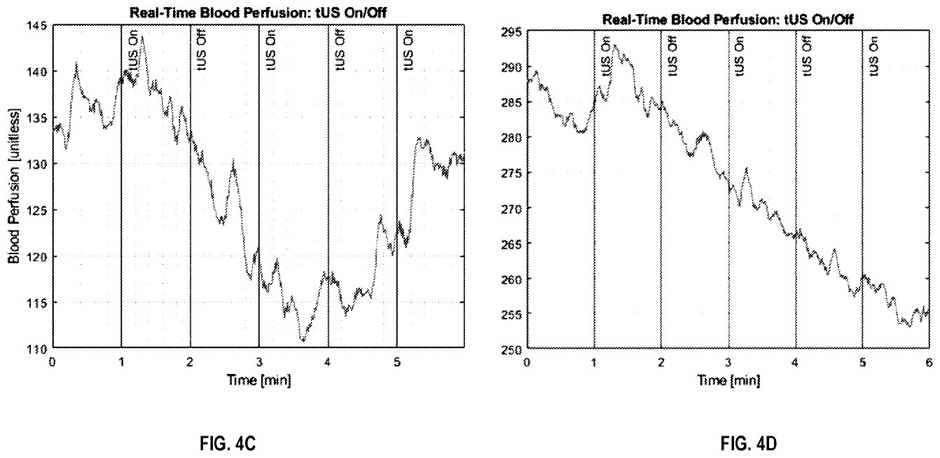

FIGS. 4A and 4B are a pair of graphical representations showing real-time blood perfusion taken during application of therapeutic ultrasound over an 11-minute period with a 5-minute “on” interval followed by a 5-minute “off” interval. FIG. 4A shows real-time blood perfusion for a full area of the object of interest observable by an optical sensor (e.g., optical sensor 20 of FIG. 2A), and FIG. 4B shows real-time blood perfusion for a region of interest (e.g., a region of the brain damaged by TBI). FIGS. 4C and 4D are another pair of graphical representations showing real-time blood perfusion taken during application of therapeutic ultrasound over a 6-minute period with alternating 1-minute “on” intervals and 1-minute “off” intervals. Likewise, FIG. 4C shows real-time blood perfusion for a full area of the object of interest observable by an optical sensor (e.g., optical sensor 20 of FIG. 2A), and FIG. 4D shows real-time blood perfusion for a region of interest (e.g., a region of the brain damaged by TBI. The photoacoustic adapter arrangements shown in FIGS. 2A-2C enable real-time observation of characteristics such as blood perfusion during therapeutic ultrasound treatment as shown in FIGS. 3A-4D.

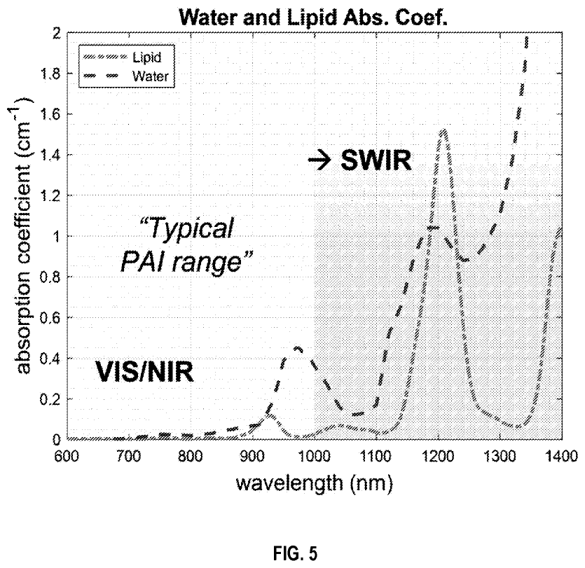

Further, as further discussed herein, the coupling medium includes gellan gum as a gelling agent and addresses technological limitations of current coupling media with respect to photoacoustic imaging. Some technologies for imaging lipids, for example, allow high contrast but only at superficial depths within tissue. There are problems with currently-available coupling media in that many of them are not suitable for photoacoustic imaging, especially for SWIR, due to properties such as acoustic attenuation and optical absorbance. FIG. 5 plots absorption coefficient for water and lipids across different wavelengths of light. Greater absorption of light in the SWIR leads to greater lipid contrast, and thus better imaging of lipids within an object of interest. At wavelengths of 1200-1400 nm, depth penetration of light is greater (as absorption by water is lower within that range) while absorption by lipids is high enough to allow high contrast in resultant imaging. Thus, sections 3 and 4 outline coupling media that aim to improve fidelity in photoacoustic imaging, especially within the SWIR range.

### 3. Coupling Medium: Gellan Gum and Hydrogen Oxide

The disclosure presents quantification of acoustic and optical properties of low acyl gellan gum as a coupling agent and compare to common coupling agents to identify the benefits and tradeoffs of each material for PAI. Properties examined in this section include acoustic attenuation, impedance, density, speed of sound, optical transmittance and optical absorbance of gellan gum at 1.5-2.5% (w/w), and compared to agarose at 1.5% (w/w), Humimic Medical Gelatin #0, and diH₂0. From the data, it is clear that gellan gum can provide an overall signal improvement of [0.43-0.86] F+0.34 dB·cm⁻¹ and [0.738-1.168] F+0.34 dB·cm⁻¹ compared to agarose and Humimic respectively, where F is the acoustic frequency in megahertz. These results suggest that low acyl gellan gum can be implemented as a coupling media for improved handheld PAI probes used for imaging skin and other clinical uses.

The goal of the validation study was to quantify the acoustic and optical properties of low acyl gellan gum (GG) and compare to common coupling agents to identify the benefits and tradeoffs of each material for PAI.

### 3.1 Materials & Methods

For the validation study, AG (QD LE Agarose), Humimic Medical Gelatin #0 (an oil-based rubber), and diH₂0 were selected as common coupling media to compare to GG. To quantify each materials coupling performance, a battery of tests were performed to determine the following properties: Optical transmittance, Optical absorbance, Speed of sound (SOS); Acoustic attenuation; Density; Acoustic impedance. Finally, to demonstrate optical performance, Light was focused through each material onto a black target, and the resulting PA signal amplitude was measured.

### 3.1-A. Sample Preparation

Sample preparations varied for each material. AG and GG start in dry powered form, and therefore must be hydrated. Preparation of these materials included steps of mixing the desired percent by weight (% w/w) powdered GG with diH₂0. This mixture was then heated and stirred until the GG has fully dissolved (~100° C.). This mixture was then degassed (~−25 inHg) to remove bubble artifacts. Finally, the resultant gel liquid was poured into the desired mold and chilled until gelling temperature (~70° C. for GG). For this study, multiple samples of GG were tested from 1.5%-2.5% (w/w) at 0.25% (w/w) increments to determine variations in optical and acoustic properties.

Likewise, preparation of AG for comparison with GG included steps of mixing the desired percent by weight (% w/w) powdered AG with diH₂0. This mixture was then heated and stirred until the AG has fully dissolved (~100° C.). This mixture was then degassed (~−25 inHg) to remove bubble artifacts. Finally, the resultant gel liquid was poured into the desired mold and chilled until gelling temperature (~36° C. for AG). AG was tested at 1.5% (w/w).

Unlike AG and GG, Humimic is an oil-based rubber and processed by the manufacturer. For preparation, Humimic was melted at ~100° C., where it was then be poured into a mold and cooled to room temperature. To remove bubble artifacts, application of an external heat gun was used.

Finally, degassed diH₂0 in a water tank was used for comparison with GG, AG, and Humimic. All solid materials were formed into identical sample dimensions (thickness=5.0 mm) using custom 3D printed molds. Each test performed had a sample size of N=4.

### 3.1-B. Mechanical Properties

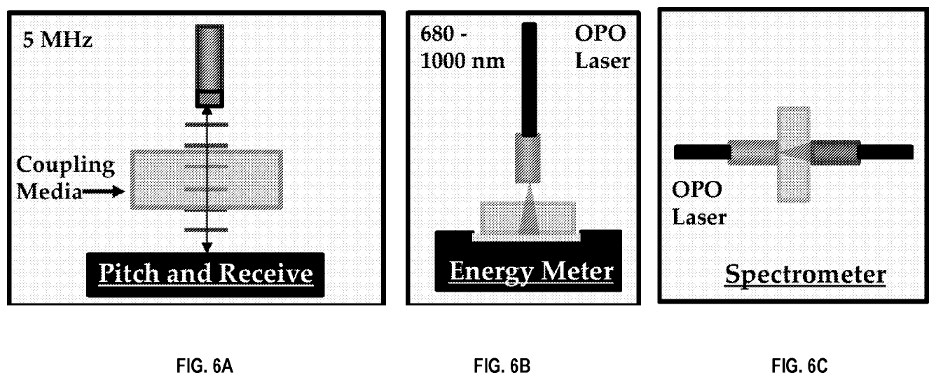

To determine SOS (c) and acoustic attenuation (a) of each material, pulse-echo ultrasound at 2.25 MHz (Panametrics A306S-SU single element) was transmitted through each material and the reflected pulse measured (FIG. 6A). Time of flight were used to calculate SOS, and overall change in reflected signal from baseline determined acoustic attenuation. Density (ρ) measurements of each materials were calculated through precise measurements of volume and mass (Kern ABT 120-4M). Finally, acoustic impedance (Z) was calculated using the SOS and density, where acoustic impedance is defined as Z=cρ.

### 3.1-C. Optical Properties

Multiple tests were performed for each material to determine their optical transmittance and absorbance. Optical transmittance was measured by sample illumination with a tunable short pulsed laser (Opolette HE 532 LD 680-1000 nm, 5 ns, 12 mJ) and an optical power meter (Coherent J-Power 1132205) (FIG. 6B). Optical absorbance was determined by placing each material in identical cuvettes and sampled from 680 nm-1000 nm with a spectrometer (Ocean Optics USB 4000 VIS/NIR) (FIG. 6C).

### 3.1-D. Photoacoustic Signal Loss

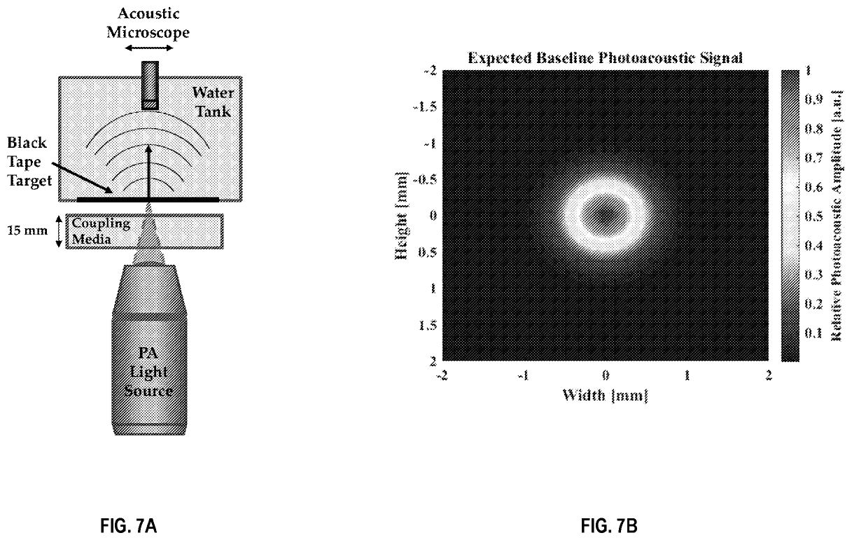

Finally, FIGS. 7A and 7B show an acoustic microscope setup for testing optical clarity of each coupling material. Short pulsed laser light was focused with a microscope object (Nikon 4/0.1) to an 800 μm Gaussian spot through 15 mm of each material onto a black target. The generated PA signal was then measured with the use of a custom acoustic microscope (Panametrics v324-sm 25 MHz) with a resolution of 200 μm (FIG. 7A). FIG. 7B shows an expected baseline photoacoustic signal measured with an acoustic microscope that the results of each coupling material are to be compared against.

### 3.2 Experimental Results

### 3.2-A. Mechanical Properties

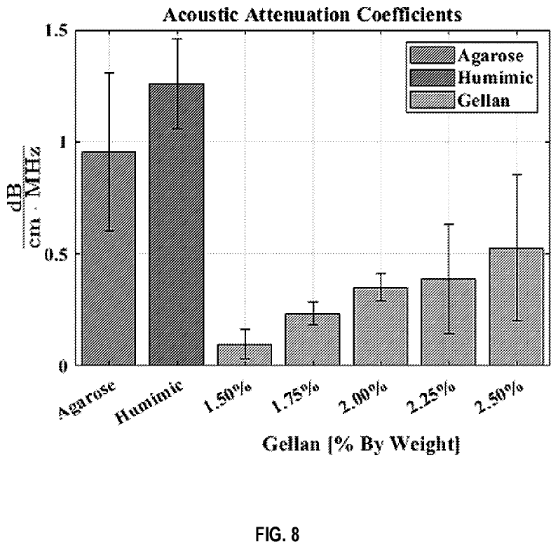

SOS, density, and acoustic impedance for all material proved similar with average values of 1469 m·s⁻¹, 0.96 g·cm³, and 1.41 MRayls respectively. The one exception was Humimic, which demonstrated significantly smaller values at 1425 m·s⁻¹, 0.78 g·cm³, and 1.1 MRayls. When comparing variations within the GG samples, GG demonstrated a linear decrease in SOS versus % w/w, with a fit value of c=−25x+1520 m·s⁻¹, where x is the % w/w of GG. Referring to FIG. 8, acoustic attenuation had larger variation between each material, with GG having considerably less overall acoustic attenuation compared to AG and Humimic. GG demonstrated a linear increase in acoustic attenuation versus % w/w, with a fit relationship of α=0.41x−0.49 dB·cm⁻¹.

### 3.2-B. Optical Properties

Upon comparison, GG showed substantially higher optical transmittance and lower absorbance compared to AG and Humimic. GG had an average transmittance of 99.3% compared to 95.6% for AG and 97.2% for Humimic. GG absorbance measurements for GG averaged at 2.23% compared to AG at 7.77% and Humimic at 3.56%. There was minimal variation in optical properties when comparing all GG samples and diH₂0.

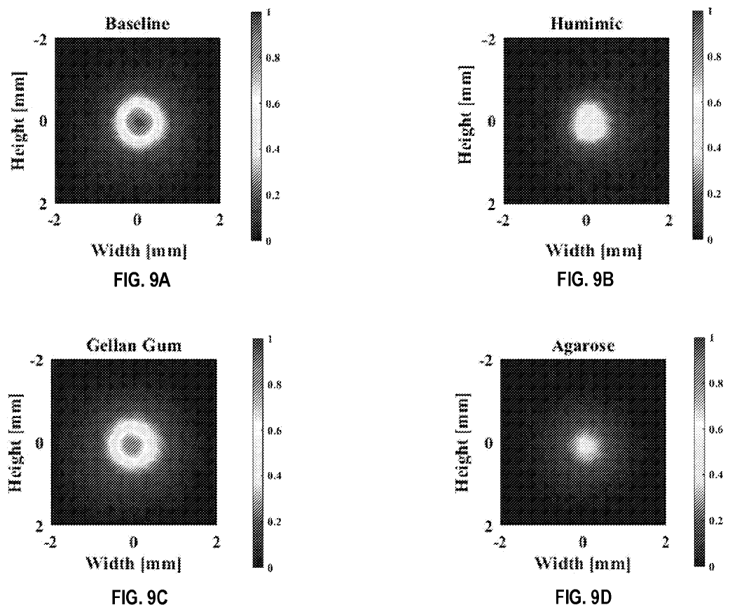

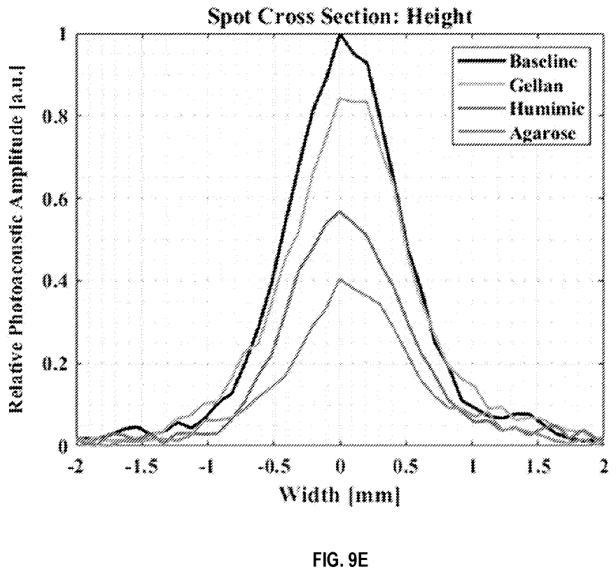

Lastly, PA images of an 800 μm Gaussian spot through each material were captured and are shown in FIGS. 9A-9D. FIG. 9A corresponds with the baseline signal shown in FIG. 7B. FIG. 9B shows a photoacoustic signal where the coupling material is Humimic, FIG. 9C shows a photoacoustic signal where the coupling material is GG, and FIG. 9D shows a photoacoustic signal where the coupling material is AG. FIG. 9E shows a “cross-sectional slice” of the relative photoacoustic amplitudes for baseline, GG, Humimic, and AG, corresponding with FIGS. 9A-9D. Comparing peak PA amplitudes, GG demonstrated significantly less PA signal loss compared to baseline at 16% loss, compared to 43% and 60% signal loss for Humimic and AG respectively.

### 3.3. Discussion

### 3.3-A. Signal Improvement

This section compares common PA coupling media with GG and quantify each material's optical and acoustic characteristics to determine coupling performance. From the data collected, GG demonstrated an improvement on optical clarity (minimal absorption and scattering) and reduction of acoustic attenuation. To determine the overall performance improvement of GG when compared to these other materials, estimations of the signal increase can be made by determining each materials total signal loss in comparison to GG.

Assuming optical loss within the medium follows the Beer-Lambert Law,

$$I = {I}_{0} {e}^{μ z} \qquad ( 1 )$$

where I is the measured Intensity, I₀ the initial energy, μ the absorption coefficient, and z the material thickness. With Eqn. 1, estimated signal improvement due to GG's optical properties can then be calculated to be:

$$\frac{1}{2} ln ( \frac{{I}_{G}}{{I}_{m}} ) \approx - 4.34 Δ μ z \qquad ( 2 )$$

where I_{g} is the light intensity measured through GG, Iₘ the compared coupling media light intensity, and Δμ the change in optical absorption coefficients. Combining Eqn. 2 with acoustic attenuation coefficients results in overall signal improvements of: [0.43-0.86] F+0.34 dB·cm⁻¹ and [0.738-1.168] F+0.34 dB·cm⁻¹ over the range of all GG samples when compared to AG and Humimic respectively, where F is the acoustic frequency in Megahertz. Due to the positive linear nature of the signal improvement versus acoustic frequency, GG will demonstrate even greater signal improvement for high frequency ultrasound when compared to AG and Humimic. This is particularly important as signal loss due to tissue attenuation is considerably more at high frequencies.

### 3.3-B. Mechanical Structure

Aside from evident PA coupling improvements, GG also demonstrated beneficial mechanical variability that is dependent on the GG concentration. At lower % w/w, GG was mechanically similar to soft tissue and deformable. However, at higher % w/w, GG became rigid and held whatever form it was molded into. This variety can allow GG to be used in a variety of experimental conditions depending on the type of coupling required.

The acoustic and optical properties of gellan gum, agarose gel, Humimic Medical Gelatin #0, and diH20 were characterized to better understand each materials ability to act as a photoacoustic coupling media. Through multiple experiments, GG was established to have significant advantages both in optical and acoustic transmission when compared to more common PA coupling media. For future work, further study into more mechanical properties such as Young's Modulus, elasticity, etc. should be studied to better characterize the material to determine ideal % w/w. Furthermore, more optical studies regarding scattering coefficients, and absorption coefficients within the visible spectrum should be studied for photoacoustic imaging at shorter wavelengths.

### 4. Coupling Medium: Gellan Gum and Deuterium Oxide

Deuterium oxide (D₂O), commonly known as “heavy water,” is an isotope of H₂O and exhibits unique and favorable properties for enabling reflection-mode PAI in the SWIR. While a few studies have demonstrated advantages of D₂O coupling for PAI, there is a lack of a thoroughly well-characterized D₂O-based gelatin to facilitate reflection-mode PAI in the SWIR. This section outlines development and assessment of the performance of a gel form of D₂O for optimal delivery of light and ultrasound to enable reflection-mode PAI in the SWIR with a penetration of several millimeters into tissue. Compared to liquid, a gelatin interface simplifies coupling to the sample for reflection-mode imaging and eliminates the potential of leakage or formation of air bubbles. A gelatin coupling medium can also be re-used and re-shaped to conform to different imaging configurations. In addition, the advantage of tunability of gelatin stiffness further provides potential for construction of impedance matching layers, which can drastically improve ultrasound propagation to and from an imaging sample.

Not solely limited to endogenous chromophores, the advancement of reflection-mode PA systems in the SWIR would also allow for detecting and monitoring exogenous contrast agents with strong and potentially tunable absorption peaks in the SWIR. This section reports a gelatin-based heavy water opto-acoustic coupling medium (“HWG”) and interface designed for reflection-mode pulse-echo (PE) and PA imaging in the SWIR. HWG is predicted to have a similar transmission profile to its liquid counterpart, which would enable in vivo PA studies above 1200 nm that require optical and acoustic coupling to a sample.

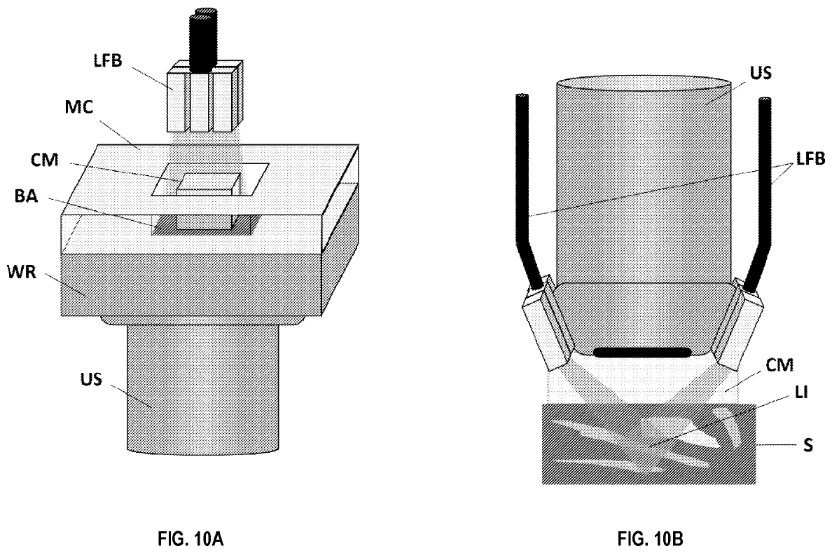

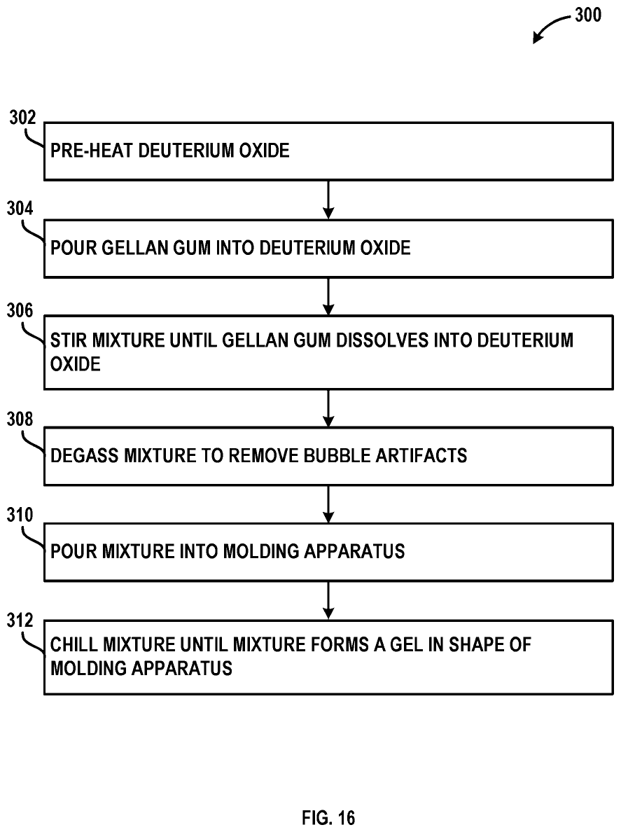

FIGS. 10A-15C show validation results for HWG as a coupling medium. FIG. 16 shows a method 300 for fabrication of HWG.

### 4.1. Methods

### 4.1-A. Fabrication of Heavy Water Gellan

Commercially acquired D₂O (United Nuclear, 99% purity) and gellan gum (Modernist Pantry, “F”, low-acyl) were used for preparing HWG. Gellan gum was chosen as the gelling agent, as opposed to standard agarose, due to its better optical transparency and efficient acoustic transmission properties as discussed in section 3 above and with respect to FIGS. 6A-9E. With reference to FIG. 16, to prepare HWG, D₂O can be pre-heated (step 302 of method 300) to ~80° C. Gellan gum powder (between 2 and 3% w/w) can be poured slowly into D₂O (step 304 of method 300) and mixed with a stir bar to ensure homogeneity (step 306 of method 300). The solution can then be degassed to remove air bubbles (step 308 of method 300) before being poured into a molding apparatus designed for PA and PE imaging (step 310 of method 300). The mixture can then be allowed to cool to room temperature until it forms a gel in the shape of the molding apparatus (step 312 of method 300), where the cooled mixture is formed into a shape conducive for insertion within a coupling medium chamber of a photoacoustic adapter. The HWG is then ready for imaging. Because the stiffness and viscosity of the resulting HWG could be altered by adjusting the concentration of gellan gum, it was determined that a concentration of 2.25% w/w gellan gum was suitable for imaging experiments because it provided mechanical stability while remaining somewhat flexible when coupling to the samples. The thickness of the HWG was ~5.0 mm for reflection-mode imaging.

### 4.1-B. Optical and Acoustic Characterization

A commercial ultrasound and PA imaging system (Vevo 3100/LAZR-X, VisualSonics) was first used in transmission-mode to quantify the optical loss through sections of HWG in the SWIR. Two small cubic samples (10×10×5 mm) of HWG were prepared (2% and 3% w/w) within a molding container illustrated in FIG. 10A (where LFB=laser fiber bundles, MC=3D printed molding container, CM=coupling medium, BA=broadband absorber/electrical tape, WR=water reservoir, US=linear array US probe). The container was placed within the output path of the fiber bundles, where acoustic coupling was then achieved from below via contact with a water reservoir and a 25 MHz US linear array (MX250, VisualSonics). Black electrical tape was used as a broadband optical absorber and inserted between the molding container and water reservoir as a baseline for estimating PA signal loss as a function of wavelength through the coupling medium. The spectrum of the tape was first measured through air in transmission-mode from 1200 to 2000 nm. The PA spectrum was then compared differentially to the broadband spectrum of the tape obtained through an optical path length defined by the thickness of HWG. Differences in these spectra result in the transmission loss corresponding to the new optical pathlength (i.e., HWG samples at 2% and 3% w/w). This method was repeated for samples of H₂O gellan gum (WG) (outlined in section 3 above) to compare the optical loss with HWG. The energy exiting the fiber bundle through the coupling media was also measured with a commercial energy meter (Coherent EnergyMax).

To determine whether HWG coupling affected acoustic propagation, the point spread function (PSF) for standard PE imaging was also calculated using a reflection-mode setup, as depicted in FIG. 10B, using samples of HWG, WG and humimic medical rubber as coupling (where LFB=laser fiber bundles, CM=coupling medium, US=linear array US probe, LI=laser illumination pattern and S=sample. Before the gel solidified, graphite powder (Loudwolf, 44 μm diameter) was distributed into the solution and allowed to cool to room temperature. These PSF images were used to estimate the axial resolution of the system through each coupling medium. Axial full-width half-max was chosen for measurement as it most closely depends on the US wavelength and dispersion (i.e., can relate to frequency-dependent attenuation of the US signal), unlike lateral and elevational resolution which can depend on additional factors, including aperture size and focal distance.

4.1-C. Sample preparation

A lipid/water phantom composed of 20% lipid shortening (Cisco), 75% diH2O, and 5% w/w agarose was prepared to demonstrate the capabilities of HWG in reflection-mode PAI operating in the SWIR compared to a WG system. The solution of lipid, water and agarose is brought to ~80° C. via hotplate, mixed with a stir bar, and left to cool and solidify at room temperature. HWG and WG coupling agents are molded for reflection-mode imaging with the 25 MHz probe and fiber bundle as described previously. Spectral PA data was collected at 5 nm intervals from 1200 to 2000 nm through both HWG and WG to quantify differences in SNR and detection. As a final validation of this reflection-mode PAI in the SWIR, a fresh sample of bovine muscle was used to demonstrate detection of intra-muscular lipid in the SWIR with HWG. Sections of locally sourced bovine tissue were cut into 10×10×10 mm cubes for full spectrum reflection mode PA and PE imaging. The PA signal amplitude as a function of depth was estimated by integrating across the lipid/water phantom in the lateral (i.e., azimuth) direction for both HWG and WG data sets.

4.2 Results

### 4.2-A. Optical and Acoustic Characterization

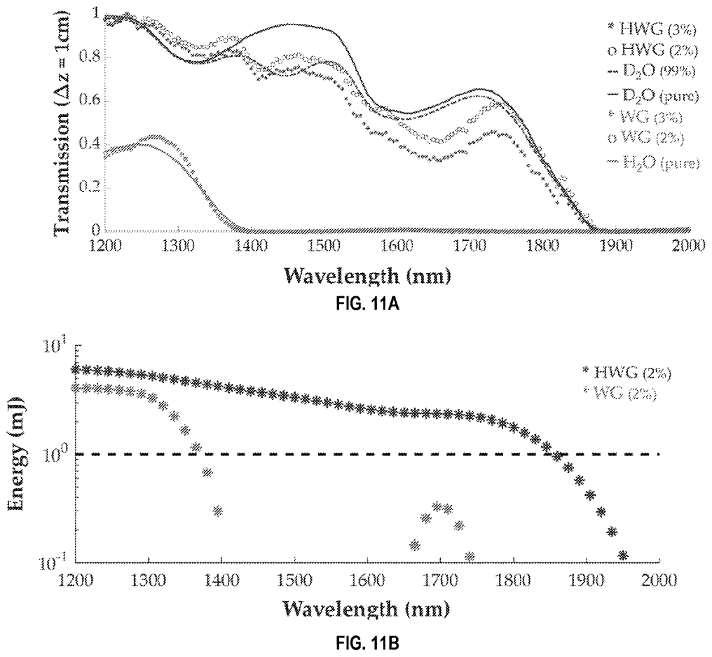

Transmission mode data is tabulated and graphed in FIGS. 11A and 11B. The relative PA signals across the band are normalized to black tape for comparing each coupling medium. It is observed that the PA transmission spectrum of HWG (2% and 3% w/w) is similar to the Beer-Lambert signal for 99% heavy water with the same optical pathlength.

Energy measurements of the LAZR-X fiber bundle through 2% w/w HWG and WG are displayed in FIGS. 11A and 11B. Criterion required that laser illumination reaching the samples with less than 1 mJ energy was insufficient for producing PA images with adequate SNR. The WG coupling agent, for example, strongly absorbed light above 1350 nm. Light delivery through HWG, on the other hand, maintained sufficient light delivery to the sample (>1 mJ) across the entire SWIR region up to 1850 nm. PA signals were too weak or undetectable outside these cutoff wavelengths.

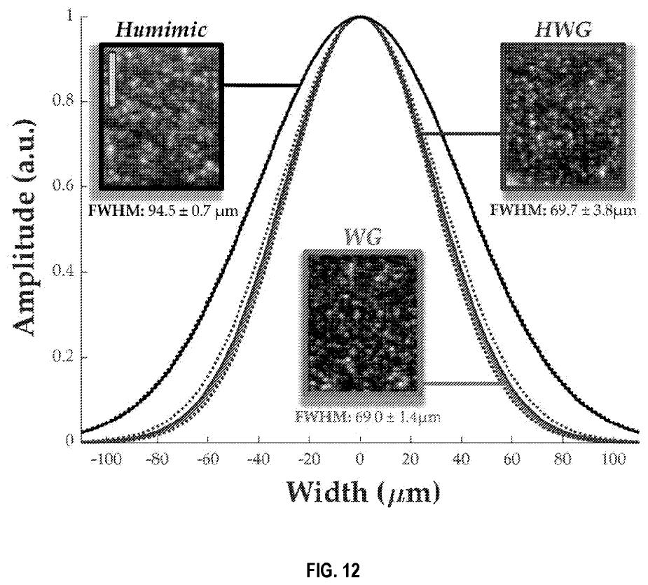

FIG. 12 describes the axial spatial resolution for PE images obtained from the sample with fine graphite particles. Analysis of the PSFs reveals similar axial spatial resolution through WG (69.0±1.4 μm) and HWG (69.7±3.8 μm), indicating that HWG preserves the full acoustic bandwidth of the propagating ultrasound waves similar to a water-based coupling agent. This is not the case with humimic rubber, as the acoustic properties are affected by dispersion and the strong attenuation at high ultrasound frequencies, resulting in a degradation in axial resolution using the 25 MHz linear array.

### 4.2-B. Lipid/Water Phantom Imaging

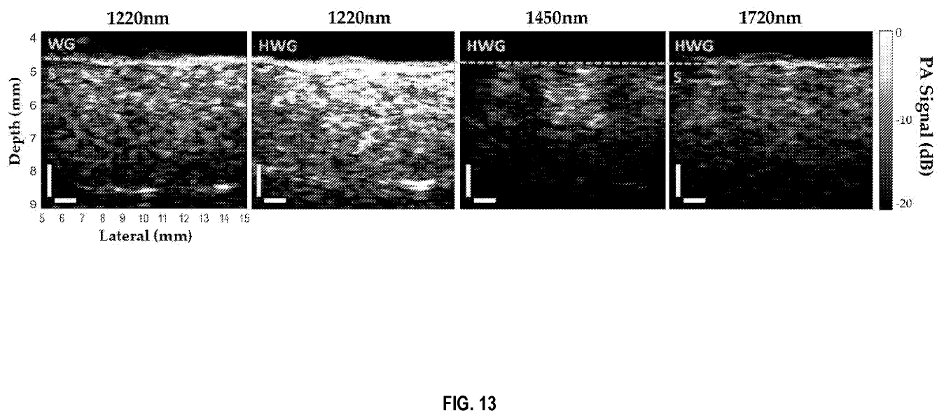

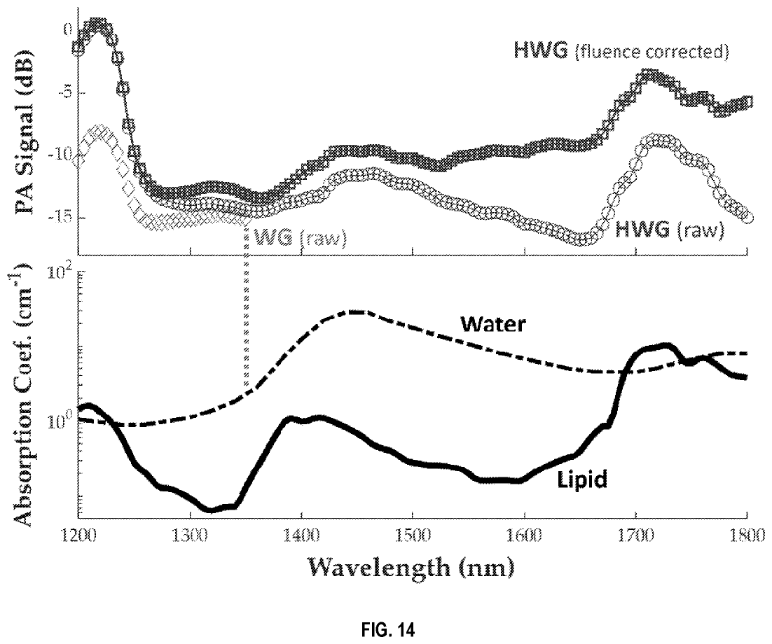

Referring to FIG. 13, PA images of the phantoms using the HWG coupling agent were obtained up to 1850 nm as predicted from the absorption coefficients and transmission measurements. On the contrary, water-based gels provided PA images of the sample up to only ~1350 nm as depicted in FIG. 13. Signals received past this WG cutoff do not contain any PA information of the sample due to insufficient light reaching the surface. PA signal above the noise floor can be seen using HWG at 1720 nm up to depths of ~5.0 mm with most of the contrast at this wavelength generated from lipids. PA surface spectra are plotted for the samples in FIG. 14, demonstrating signals above noise for HWG across this full range, including distinct peaks of lipid/water.

At 1220 nm, a 7.5 dB signal increase is observed at the surface of the phantom using HWG compared to WG. This agrees closely with what is expected via the absorption plots in FIGS. 11A and 11B through a similar optical pathlength at 1220 nm. At a depth of 4.5 mm into the sample, a 4.6 dB signal increase is observed, indicating at depth an SNR increase of nearly 2× is apparent with HWG coupling compared to WG coupling at shorter SWIR wavelengths. For this sample, the noise floor is reached through HWG at a depth of ~5 mm. At 1720 nm, a 24 dB increase in maximum surface signal is observed when imaged through HWG and fluence corrected as opposed to WG (at the noise floor at this wavelength due to insufficient light delivery to the sample), illustrating the extended usable wavelength range for HWG-enabled PA systems when compared to standard coupling methods.

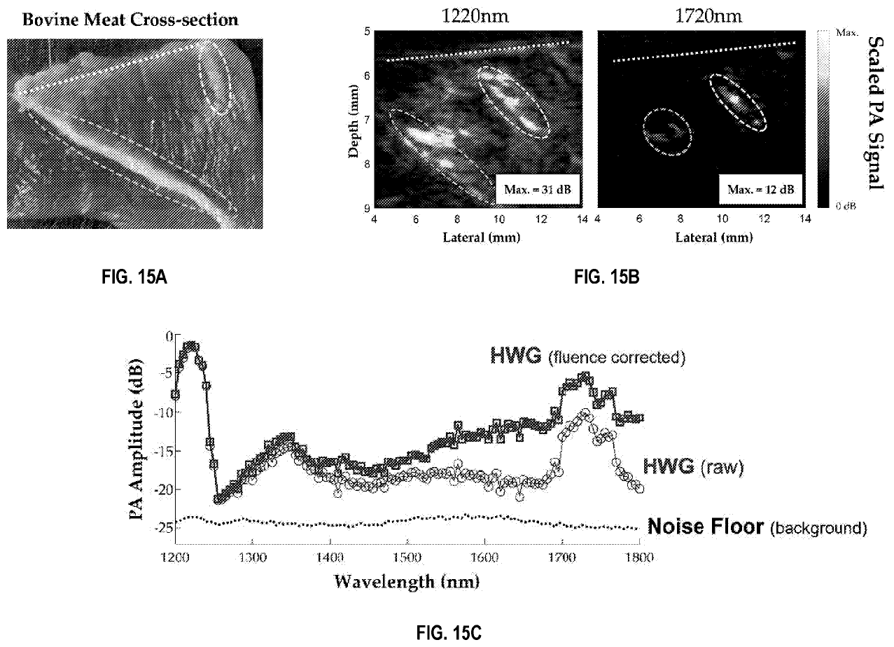

FIGS. 15A-15C display a PA imaging cross-section of the bovine tissue sample along with spectral data. Even at 1220 nm (obtainable with WG), the ratio of peak PA signal in the green and yellow regions of interest (ROI) between the HWG and WG images is 3.04× and 1.41× respectively, indicating a broad increase in SNR when using HWG coupling as opposed to WG.

### 4.3 Discussion

HWG enables PAI of tissue samples across a broad spectral range in the SWIR (1200-1850 nm), whereas WG is limited to wavelengths<1350 nm with poor SNR (FIGS. 11A and 11B). It was anticipated that the predicted transmission spectrum of HWG would be similar to that of heavy water in liquid form at the equivalent concentration (99% pure). The results align with this prediction, as illustrated in FIGS. 11A and 11B. Slight shifts in the transmission peaks in the WG sample compared to baseline are likely due to the bonding mechanism of the low-acyl gellan gum, which has been reported previously. This effect is also observed with HWG, indicating the bonding effects of gellan and heavy water are similar to that of WG. Akin to water-based coupling, HWG has minimal loss of high frequency acoustic waves, preserving spatial resolution for PE imaging as shown in FIG. 12. HWG enables the study of tissue constituents in reflection-mode PA setups without sacrificing spatial resolution. Oil-based rubbers like humimic gels have much stronger acoustic attenuation at high ultrasound frequencies (>20 MHz) due to dispersion and absorption. In the context of FIG. 12, this implies the PSF produced for humimic gel should be wider in spatial width, which is corroborated with results using the 25 MHz linear array. The near identical results for HWG and WG signify no loss of PE axial resolution (~70 μm) through the coupling medium, which is necessary for HWG to be applied to studies with simultaneous high-specificity and high-resolution mapping of constituents, including dual modality PE and PA imaging and spectroscopy in the SWIR. Finally, reflection mode PAI was demonstrated in the SWIR using the HWG as a solid coupling medium designed for in vivo imaging on a commercial scanning system. The HWG interface provides a considerable boost in SNR with penetration into the samples of ~5.0 mm at 1720 nm. Lipid pool regions in ex vivo bovine tissue exhibit a >12 dB increase in the PA signal at 1220 and 1720 nm. This further demonstrates the potential impact of the HWG interface for in vivo reflection mode ultrasound PA imaging studies for detecting and tracking biomarkers like LWC, which have strong and unique optical absorption signatures in the SWIR.

The high-resolution, high-specificity study of tissue constituents such as LWC can henceforth be aided by the addition of HWG to standard reflection-mode PA systems. Current and past systems used to study constituents may find that the implementation of HWG will result in improved SNR, along with allowing for a larger usable optical bandwidth for spectral characterization and/or unmixing. Other designs which would benefit from improved opto-acoustic coupling include fiber bundle arrangements and optoacoustic in-line reflectors that generally require coupling media. A limitation of the validation study was that no direct validation with histology was performed to quantify the presence of LWC. Studies which implement spectral-unmixing algorithms to identify LWC and other constituents in tissue may find a benefit in the increased SNR at chosen wavelengths provided through HWG; however, future validation studies are needed to determine the accuracy of these HWG-enabled spectral unmixing algorithms in the SWIR.

Furthermore, clutter effects of PA-induced US waves launched from the sample surface were not explored. This phenomenon is well documented and known to add discrepancies in PA images which are not representative of standard PA contrast mechanisms. To further characterize HWG's potential as a reflection-mode coupling medium, future research should delve into the effects of PA-induced US waves on tissue as a function of coupling absorption. Future work will also include the 3D capability of commercial systems like the Vevo LAZR-X to obtain 3D PA and PE volume data with the HWG retaining contact with the tissue sample or animal. Reflection-mode PA systems with increased capabilities also have promise in clinical environments to aid in the diagnosis of superficial skin lesions relating to skin cancer or tracking the progression of wound healing. Efforts are underway to incorporate HWG into a novel closed-loop reflection-mode PA system tailored for imaging skin lesions and monitoring wound healing. A patent application has been issued for the HWG interface mechanism as described in this manuscript. Along with obvious general applications towards PAI, HWG may be used in ultrasound modulation therapy studies; HWG implemented within a proper system design could enable real-time and efficient imaging for changes in function during ultrasound modulation therapy. This could prove useful for treating several medical conditions ranging from traumatic brain injury and stroke to peripheral neuropathy.

The particular implementations shown and described are illustrative of the technology and its best mode and are not intended to otherwise limit the scope of the present technology in any way. Indeed, for the sake of brevity, conventional manufacturing, connection, preparation, and other functional aspects of the apparatus may not be described in detail. Furthermore, the connecters and points of contact shown in the various figures are intended to represent exemplary physical relationships between the various elements. Many alternative or additional functional relationships or physical connections may be present in a practical system.

In the foregoing description, the technology has been described with reference to specific exemplary embodiments. Various modifications and changes may be made, however, without departing from the scope of the present technology as set forth. The description and figures are to be regarded in an illustrative manner, rather than a restrictive one and all such modifications are intended to be included within the scope of the present technology. Accordingly, the scope of the technology should be determined by the generic embodiments described and their legal equivalents rather than by merely the specific examples described above. For example, the steps recited in any method or process embodiment may be executed in any appropriate order and are not limited to the explicit order presented in the specific examples. Additionally, the components and/or elements recited in any system embodiment may be combined in a variety of permutations to produce substantially the same result as the present technology and are accordingly not limited to the specific configuration recited in the specific examples.

Benefits, other advantages, and solutions to problems have been described above with regard to particular embodiments. Any benefit, advantage, solution to problems or any element that may cause any particular benefit, advantage, or solution to occur or to become more pronounced, however, is not to be construed as a critical, required, or essential feature or component.

The present technology has been described above with reference to an exemplary embodiment. However, changes and modifications may be made to the exemplary embodiment without departing from the scope of the present technology. These and other changes or modifications are intended to be included within the scope of the present technology.

1. A photoacoustic adapter for real-time optical monitoring using ultrasound, including:
a coupling medium chamber defining:
a first optical window along a proximal portion of the coupling medium chamber;
a second optical window along a distal portion of the coupling medium chamber and configured to contact a surface of an object of interest, the second optical window being defined opposite from the first optical window, the first optical window and the second optical window being coaligned along a first axis;
an ultrasound transducer port configured to engage an ultrasonic probe;
a coupling medium disposed within the coupling medium chamber, the coupling medium including a gelling agent; and
a prism element disposed within the coupling medium chamber and configured to direct acoustic emissions from the ultrasonic probe towards the object of interest in alignment with the first axis;
wherein a response of the object of interest to the acoustic emissions is observable by an optical sensor aligned with the first optical window.

2. The photoacoustic adapter of claim 1, the gelling agent of the coupling medium including gellan gum.

3. The photoacoustic adapter of claim 2, the coupling medium further including water.

4. The photoacoustic adapter of claim 2, the coupling medium further including deuterium oxide.

5. The photoacoustic adapter of claim 1, wherein the coupling medium includes low acyl gellan gum having a concentration of 1.5% w/w to 3% w/w.

6. An adapter for use with an ultrasonic probe; the adapter comprising:
a proximal portion configured to engage an ultrasonic probe;
a distal portion configured to contact a surface of an object of interest; and
a coupling medium chamber disposed between the proximal portion and the distal portion and including a coupling medium, the coupling medium including a gelling agent.

7. The adapter of claim 6, the gelling agent of the coupling medium including gellan gum.

8. The adapter of claim 7, the coupling medium further including water.

9. The adapter of claim 6, the coupling medium further including deuterium oxide.

10. The adapter of claim 6, wherein the coupling medium includes low acyl gellan gum having a concentration of 1.5% w/w to 3% w/w.

11. The adapter of claim 6, wherein the adapter is configured to co-align optical transmissions from the ultrasonic probe with acoustic emissions received from the object of interest at the ultrasonic probe.

12. A coupling medium for use with an ultrasonic probe, the coupling medium comprising gellan gum.

13. The coupling medium of claim 12, further comprising deuterium oxide.

14. The coupling medium of claim 12, further comprising water.

15. The coupling medium of claim 12, wherein the coupling medium includes low acyl gellan gum having a concentration of 1.5% w/w to 3% w/w.

16. The coupling medium of claim 12, wherein the coupling medium is molded to a shape conducive for insertion within a coupling medium chamber in association with the ultrasonic probe.

17. A method of fabricating a coupling medium, comprising:
mixing gellan gum with deuterium oxide to form a mixture;
degassing the mixture to remove bubble artifacts from the mixture;
pouring the mixture into a molding apparatus; and
chilling the mixture until the mixture forms a gel in a shape of the molding apparatus.

18. The method of claim 17, the gellan gum being in dry powder form, wherein mixing gellan gum with deuterium oxide to form the mixture includes:
pre-heating the deuterium oxide;
pouring gellan gum into the deuterium oxide; and
stirring the mixture until the gellan gum dissolves into the deuterium oxide.

19. The method of claim 17, the gellan gum being low acyl gellan gum having a concentration of 1.5% w/w to 3% w/w.

20. The method of claim 17, where the molding apparatus is configured to form the mixture into a shape conducive for insertion within a coupling medium chamber of a photoacoustic adapter.
